(19) United States
(12) Patent Application Publication
(10) Pub. No.: US 2026/0245265 A1
(43) Pub. Date: Aug. 20, 2026

(54) DEVICE AND METHOD FOR ANALYZING EMOTIONS AND HUMAN RELATIONSHIPS USING COLORS AND A COMPUTER PROGRAM THEREFOR

(71) Applicant: Jung Wook KIM, Seoul (KR)

(72) Inventors: Jung Wook KIM, Seoul (KR); Soo-Kyoung LEE, Seoul (KR); Eun-Ok KIM, Goyang-si (KR); Su-Yeon KIM, Seoul (KR); Seung-Hee HONG, Seoul (KR)

(21) Appl. No.: 19/306,393

(22) Filed: Aug. 21, 2025

(30) Foreign Application Priority Data
Feb. 17, 2025 (KR) 10-2025-0020162

(51) Int. Cl. G06T 11/00; G06T 7/90

(52) U.S. Cl. CPC G06T 11/10; G06T 7/90; G06T 2200/24; G06T 2207/10024

(57) ABSTRACT

Disclosed is a device and method for analyzing an emotion and a human relationship based on a color. The device includes a display; and a processor electrically connected to the display, wherein the processor is configured to: display an application for analyzing an emotion and a human relationship based on a color on the display; identify a color corresponding to a user, a color corresponding to an age group of the user, a color corresponding to each of emotions of the user, and a color corresponding to an acquaintance of the user as input from the user through the displayed application; extract RGB values of each of the identified colors, convert the extracted RGB values into three-dimensional color coordinates, and display the converted three-dimensional color coordinates on the application.

### CROSS-REFERENCE TO RELATED APPLICATION

This application claims priority from Korean Patent Application No. 10-2025-0020162 filed on Feb. 17, 2025 in the Korean Intellectual Property Office, and all the benefits accruing therefrom under 35 U.S.C. 119, the contents of which in its entirety are herein incorporated by reference.

### BACKGROUND

### Field

The present disclosure relates to a device and a method for analyzing emotions and human relationships based on colors, and a computer program therefor.

### Description of Related Art

Conventionally, an existing analysis method for analyzing emotional, psychological changes, and human relationships was mainly based on language. However, this method may have different results related to some users depending on the user's ability to express language or due to differences in linguistic understanding of the users, or the accuracy of the analysis thereof may be reduced.

The prior art documents (Korean Patent Application Publication No. 10-2024-0083400 and 2024.06.12) relates to a system for providing psychological analysis and healing information using color and aroma, and discloses a scheme of allowing a user's emotional state to be analyzed using a color emotion card using a color to increase the accuracy of analysis and recommending aroma capable of healing the analyzed emotional state.

However, the prior art document merely discloses outputting color emotion cards of various colors on which words related to emotions are written and allowing a user to select one therefrom and recommending the aroma based on selected one. The prior art fails to disclose that when the user selects his/her own color, a color corresponding to each of his/her various emotions, a color corresponding to an age group of the user, a color corresponding to an acquaintance of the user, etc., a similarity between the color corresponding to the user and each of the color of the emotion of the user, the color corresponding to the age group, and the color corresponding to the emotion thereof relative to the acquaintance is analyzed, and the analysis result is provided using 3-dimensional color coordinates.

### PRIOR ART LITERATURE

Patent document
Patent Document 1: Korean Patent Application Publication 10-2024-0083400 (2024.06.12)

### SUMMARY

Accordingly, a technique is needed in which when the user selects his/her own color, a color corresponding to each of his/her various emotions, a color corresponding to an age group of the user, a color corresponding to an acquaintance of the user, etc., a similarity between the color corresponding to the user and each of the color of the emotion of the user, the color corresponding to the age group, and the color corresponding to the emotion thereof relative to the acquaintance is analyzed, and the analysis result is provided using 3-dimensional color coordinates so that the user may intuitively check his/her emotional state.

Thus, a purpose of the present disclosure is to provide a device and method for analyzing emotions and human relationships based on colors, and a computer program therefor.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims or combinations thereof.

A first aspect of the present disclosure provides a device for analyzing an emotion and a human relationship based on a color, the device comprising: a display; and a processor electrically connected to the display, wherein the processor is configured to: display an application for analyzing an emotion and a human relationship based on a color on the display; identify a color corresponding to a user, a color corresponding to an age group of the user, a color corresponding to each of emotions of the user, and a color corresponding to an acquaintance of the user as input from the user through the displayed application; extract RGB values of each of the identified colors, convert the extracted RGB values into three-dimensional color coordinates, and display the converted three-dimensional color coordinates on the application; convert the converted three-dimensional color coordinates into three-dimensional solar system orbits and display the converted three-dimensional solar system orbits on the application; and analyze the user's emotion, the age group of the user, and a relationship between the user and the acquaintance, based on the converted three-dimensional solar system orbits and display the analyzed result on the display.

In accordance with some embodiments of the device, the processor is further configured to convert the extracted RGB values into L*, a*, and b* values, respectively, based on a Cielab conversion formula.

In accordance with some embodiments of the device, the processor is further configured to: calculate a distance between the color corresponding to the user and the color corresponding to each of emotions of the user, on the three-dimensional color coordinates; and apply the calculated distance to the three-dimensional color coordinates and visually display the resulting three-dimensional color coordinates on the application.

In accordance with some embodiments of the device, the processor is further configured to: when the color corresponding to the user and the color corresponding to each of emotions are input from the user, calculate a distance between the color corresponding to the user and the color corresponding to each of emotions of the user; and analyze a similarity between the color corresponding to the user and the color corresponding to each of emotions of the user, based on the calculated distance.

In accordance with some embodiments of the device, the processor is further configured to: when the color corresponding to each of emotions of the user and the color corresponding to the age group of the user are input from the user, calculate a distance between the color corresponding to each of emotions of the user and the color corresponding to the age group of the user; and analyze a similarity between the color of each of emotions of the user and the color corresponding to the age group of the user, based on the calculated distance.

In accordance with some embodiments of the device, the processor is further configured to: when the color corresponding to each of emotions of the user and the color corresponding to the acquaintance are input from the user, calculate a distance between the color corresponding to each of emotions of the user and the color corresponding to the acquaintance; and analyze a similarity between the color corresponding to each of emotions of the user and the color corresponding to the acquaintance, based on the calculated distance.

In accordance with some embodiments of the device, as the calculated distance is smaller, the similarity is higher, wherein as the calculated distance is greater, the similarity is lower.

A second aspect of the present disclosure provides a method for analyzing an emotion and a human relationship based on a color, the method comprising: displaying an application for analyzing an emotion and a human relationship based on a color on a display; identifying a color corresponding to a user, a color corresponding to an age group of the user, a color corresponding to each of emotions of the user, and a color corresponding to an acquaintance of the user as input from the user through the displayed application; extracting RGB values of each of the identified colors, converting the extracted RGB values into three-dimensional color coordinates, and displaying the converted three-dimensional color coordinates on the application; converting the converted three-dimensional color coordinates into three-dimensional solar system orbits and displaying the converted three-dimensional solar system orbits on the application; and analyzing the user's emotion, the age group of the user, and a relationship between the user and the acquaintance, based on the converted three-dimensional solar system orbits and displaying the analyzed result on the display.

In accordance with some embodiments of the method, extracting the RGB values each of the identified colors, converting the extracted RGB values into the three-dimensional color coordinates, and displaying the converted three-dimensional color coordinates on the application include: calculating a distance between the color corresponding to the user and the color corresponding to each of emotions of the user, on the three-dimensional color coordinates; and applying the calculated distance to the three-dimensional color coordinates and visually displaying the resulting three-dimensional color coordinates on the application.

In accordance with some embodiments of the method, analyzing the user's emotion, the age group of the user, and the relationship between the user and the acquaintance includes: when the color corresponding to the user and the color corresponding to each of emotions are input from the user, calculating a distance between the color corresponding to the user and the color corresponding to each of emotions of the user; and analyzing a similarity between the color corresponding to the user and the color corresponding to each of emotions of the user, based on the calculated distance.

In accordance with some embodiments of the method, analyzing the user's emotion, the age group of the user, and the relationship between the user and the acquaintance includes: when the color corresponding to each of emotions of the user and the color corresponding to the age group of the user are input from the user, calculating a distance between the color corresponding to each of emotions of the user and the color corresponding to the age group of the user; and analyzing a similarity between the color of each of emotions of the user and the color corresponding to the age group of the user, based on the calculated distance.

In accordance with some embodiments of the method, analyzing the user's emotion, the age group of the user, and the relationship between the user and the acquaintance includes: when the color corresponding to each of emotions of the user and the color corresponding to the acquaintance are input from the user, calculating a distance between the color corresponding to each of emotions of the user and the color corresponding to the acquaintance; and analyzing a similarity between the color corresponding to each of emotions of the user and the color corresponding to the acquaintance, based on the calculated distance.

A third aspect of the present disclosure provides a computer program stored in a computer readable medium, wherein the computer program stores therein instructions for analysis of an emotion and a human relationship based on a color, wherein when the instructions are executed by a processor, the instructions cause the processor to: display an application for analyzing an emotion and a human relationship based on a color on a display; identify a color corresponding to a user, a color corresponding to an age group of the user, a color corresponding to each of emotions of the user, and a color corresponding to an acquaintance of the user as input from the user through the displayed application; extract RGB values of each of the identified colors, convert the extracted RGB values into three-dimensional color coordinates, and display the converted three-dimensional color coordinates on the application; convert the converted three-dimensional color coordinates into three-dimensional solar system orbits and display the converted three-dimensional solar system orbits on the application; and analyze the user's emotion, the age group of the user, and a relationship between the user and the acquaintance, based on the converted three-dimensional solar system orbits and display the analyzed result on the display.

According to the present disclosure, when the user selects the color corresponding to the user and the color corresponding to each of the emotions such as anxiety, nervousness, pleasure, sadness, anger, joy, and fullness, the device may be configured to analyze the similarity between the selected color corresponding to the user and the selected color corresponding to each of the emotions such as anxiety, nervousness, pleasure, sadness, anger, joy, and fullness, and visually display the similarity on the application, so that the user may intuitively grasp the current state of the user related to the various emotions.

Further, when the user selects the color corresponding to the age group and the color corresponding to each of the emotions such as anxiety, nervousness, pleasure, sadness, anger, joy, and fullness, the device may be configured to calculate the similarity between the color corresponding to the age group selected by the user and the selected color corresponding to each of the emotions such as anxiety, nervousness, pleasure, sadness, anger, joy, and fullness selected by the user and visually display the similarity on the application. Thus, the user may intuitively grasp the current state of the user with respect to various emotions based on his/her age group.

Moreover, when the user selects the color corresponding to the acquaintance and the color corresponding to each of the emotions such as anxiety, nervousness, pleasure, sadness, anger, joy, and fullness, the device may be configured to calculate the similarity between the color corresponding to the acquaintance selected by the user and the color corresponding to each of the emotions such as anxiety, nervousness, pleasure, sadness, anger, joy, and fullness selected by the user and visually display the similarity on the application. Thus, the user may intuitively grasp the current state of various emotions of the user with respect to the acquaintance.

Effects of the present disclosure are not limited to the effects mentioned above, and other effects not mentioned will be clearly understood by those skilled in the art from the description as set forth below.

In addition to the above effects, specific effects of the present disclosure are described together while describing specific details for carrying out the present disclosure.

### BRIEF DESCRIPTION OF DRAWINGS

FIG. 1 is a block diagram illustrating an device for analyzing an emotion and a human relationship based on a color according to an embodiment of the present disclosure.

FIG. 2 is a flowchart illustrating a process of analyzing emotions and human relationships based on colors according to an embodiment of the present disclosure.

(a) in FIG. 3 is an example diagram illustrating each of emotions of a user based on a color according to an embodiment of the present disclosure.

(b) in FIG. 3 is an example diagram illustrating an age group of a user based on a color according to an embodiment of the present disclosure.

(c) in FIG. 3 is an example diagram illustrating a color corresponding to an acquaintance of a user according to an embodiment of the present disclosure.

FIG. 4 is an example diagram illustrating a three-dimensional color coordinate according to an embodiment.

FIG. 5 is an example diagram illustrating a three-dimensional solar system orbits according to an embodiment of the present disclosure.

FIG. 6 is an example diagram illustrating an emotion state of a user according to an embodiment of the present disclosure.

FIG. 7A is an example diagram illustrating an emotion state of a user when the user is in his/her teens according to an embodiment of the present disclosure.

FIG. 7B is an example diagram illustrating an emotion state of a user when the user is in his/her twenties according to an embodiment of the present disclosure.

FIG. 7C is an example diagram illustrating an emotion state of a user when the user is in his/her thirties according to an embodiment of the present disclosure.

FIG. 7D is an example diagram illustrating an emotion state of a user when the user is in his/her forties according to an embodiment of the present disclosure.

FIG. 8A is an example diagram illustrating an emotion state of a user with respect to a father according to an embodiment of the present disclosure.

FIG. 8B is an example diagram illustrating an emotion state of a user with respect to a mother according to an embodiment of the present disclosure.

FIG. 8C is an example diagram illustrating an emotion state of a user with respect to an older sister according to an embodiment of the present disclosure.

FIG. 8D is an example diagram illustrating an emotion state of a user with respect to a younger brother according to an embodiment of the present disclosure.

### DETAILED DESCRIPTIONS

Advantages and features of the present disclosure, and a method of achieving the advantages and features will become apparent with reference to embodiments described below in detail together with the accompanying drawings. However, the present disclosure is not limited to the embodiments as disclosed under, but may be implemented in various different forms. Thus, these embodiments are set forth only to make the present disclosure complete, and to entirely inform the scope of the present disclosure to those of ordinary skill in the technical field to which the present disclosure belongs, and the present disclosure is only defined by the scope of the claims.

For simplicity and clarity of illustration, elements in the drawings are not necessarily drawn to scale. The same reference numbers in different drawings represent the same or similar elements, and as such perform similar functionality. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

A shape, a size, a ratio, an angle, a number, etc. disclosed in the drawings for illustrating embodiments of the present disclosure are illustrative, and the present disclosure is not limited thereto.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this disclosure, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify an entirety of the list of elements and may not modify the individual elements of the list.

In interpretation of numerical values, an error or tolerance therein may occur even when there is no explicit description thereof.

It will be understood that when a first element or layer is referred to as being “connected to”, or “coupled to” a second element or layer, the first element may be directly connected to or coupled to the second element or layer, or one or more intervening elements or layers may be present therebetween.

In descriptions of temporal relationships, for example, temporal precedent relationships between two events such as “after”, “subsequent to”, “before”, etc., another event may occur therebetween unless “directly after”, “directly subsequent” or “directly before” is not indicated.

When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

It will be understood that, although the terms “first”, “second”, “third”, and so on may be used herein to describe various elements, components, areas, layers and/or periods, these elements, components, areas, layers and/or periods should not be limited by these terms. These terms are used to distinguish one element, component, area, layer or section from another element, component, area, layer or section. Thus, a first element, component, area, layer or section as described under could be termed a second element, component, area, layer or section, without departing from the spirit and scope of the present disclosure.

When an embodiment may be implemented differently, functions or operations specified within a specific block may be performed in a different order from an order specified in a flowchart. For example, two consecutive blocks may actually be performed substantially simultaneously, or the blocks may be performed in a reverse order depending on related functions or operations.

The features of the various embodiments of the present disclosure may be partially or entirely combined with each other, and may be technically associated with each other or operate with each other. The embodiments may be implemented independently of each other and may be implemented together in an association relationship.

In interpreting a numerical value, the value is interpreted as including an error range unless there is no separate explicit description thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “embodiments,” “examples,” “aspects, etc. should not be construed such that any aspect or design as described is superior to or advantageous over other aspects or designs. Further, the term ‘or’ means ‘inclusive or’ rather than ‘exclusive or’. That is, unless otherwise stated or clear from the context, the expression that ‘x uses a or b’ means one of natural inclusive permutations.

The terms used in the description as set forth below have been selected as being general and universal in the related technical field. However, there may be other terms than the terms depending on the development and/or change of technology, convention, preference of technicians, etc. Therefore, the terms used in the description as set forth below should not be understood as limiting technical ideas, but should be understood as examples of the terms for illustrating embodiments. Further, in a specific case, a term may be arbitrarily selected by the applicant, and in this case, the detailed meaning thereof will be described in a corresponding description period. Therefore, the terms used in the description as set forth below should be understood based on not simply the name of the terms, but the meaning of the terms and the contents throughout the Detailed Descriptions.

In description of flow of a signal, for example, when a signal is delivered from a node A to a node B, this may include a case where the signal is transferred from the node A to the node B via another node unless a phrase ‘immediately transferred’ or ‘directly transferred’ is used. Throughout the present disclosure, “A and/or B” means A, B, or A and B, unless otherwise specified, and “C to D” means C inclusive to D inclusive unless otherwise specified.

Hereinafter, a device and method for analyzing an emotion and a human relationship based on a color according to some embodiments of the present disclosure will be described.

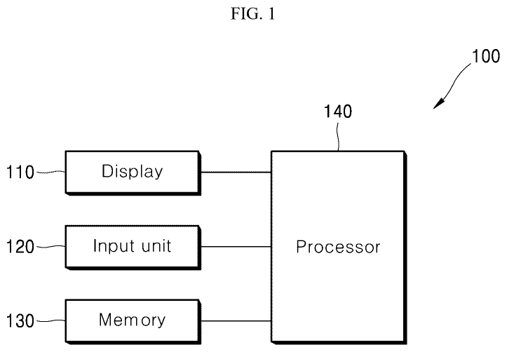

FIG. 1 is a block diagram illustrating an device for analyzing an emotion and a human relationship based on a color according to an embodiment of the present disclosure.

Referring to FIG. 1, an device 100 for analyzing and expressing an emotion and a human relationship based on a color according to an embodiment of the present disclosure may include a display 110, an input unit 120, a memory 130, and a processor 140.

The configuration of the device 100 illustrated in FIG. 1 is merely one embodiment, and the components of the device 100 are not limited to the embodiment illustrated in FIG. 1, and some components may be added, changed, or deleted as necessary.

According to an embodiment, the display 110 may display an application that analyzes and expresses the user's emotion and human relationship based on colors. The application may receive, from the user, a color corresponding to the user (that is, a color corresponding to a user's temperament, personality, or disposition), a color corresponding to a user's age group (e.g., 10s, 20s, 30s, 40s, etc.), a color corresponding to a user's emotion (e.g., joy, anger, sadness, pleasure, anxiety, nervousness, fullness, etc.), and a color corresponding to a user's acquaintance (e.g., father, mother, older sister, older brother, younger sister, younger brother, etc.) (that is, a color corresponding to temperament, personality, or disposition of the user's acquaintance) through the input unit 120. For example, the display 110 may include a touch sensing circuit for sensing a touch input.

According to an embodiment, the display 110 may display a three-dimensional color coordinate and/or three-dimensional solar system orbits based on a color corresponding to the user, a color corresponding to the user's age group, a color corresponding to each of emotions of the user, and a color of each of a user's acquaintances as input through the application. In addition, the display 110 may display an analysis result of analyzing the user's emotion, the age group thereof, and a relationship between the user and the color corresponding to the acquaintance, based on the three-dimensional solar system orbits.

According to an embodiment, the input unit 120 may include a keyboard, a mouse, a joystick, etc. Using the input unit 120, the user may select a color corresponding to each of the user's age groups (e.g., 10s, 20s, 30s, 40s, etc.), a color corresponding to each of the emotions (e.g., joy, anger, sadness, pleasure, anxiety, novelty, fullness, etc.) of the user, a color corresponding to the user (i.e., a color corresponding to the temperament, personality, or disposition of the user), a color corresponding to the acquaintance of the user (i.e., a color corresponding to the temperament, personality, or disposition of the acquaintance) and may input the selected ones into the application.

According to an embodiment, the memory 130 may store therein the application that analyzes and expresses the user's emotion and human relationship based on a color. In addition, the memory 130 may store therein various information input from the user.

In addition, the memory 130 may store therein instructions or a program for analyzing and expressing the emotion and human relationship of the user based on a color.

According to an embodiment, the processor 140 may be configured to generally control the components (e.g., the display 110, the input unit 120, and the memory 130) of the device 100.

For example, the processor 140 may be embodied as at least one physical element selected from among application specific integrated circuits (ASICs), digital signal processors (DSPs), digital signal processing devices (DSPDs), programmable logic devices (PLDs), field programmable gate arrays (FPGAs), processors, micro-controllers, and microprocessors.

The processor 140 may be configured to include an artificial intelligence algorithm. The artificial intelligence algorithm may be implemented using the processor 140. The artificial intelligence algorithm refers to a program that mimics the human brain neural network, and may support a deep learning algorithm that analyzes, perceives, inferences, and determines various data by itself, and thus may mathematically calculate and analyze/express human emotions and human relationships of the user based on the color.

According to an embodiment, the processor 140 may be configured to display the application for analyzing and expressing various emotions of a user and a human relationship related to the user based on a color through the display 110.

According to an embodiment, the processor 140 may be configured to obtain information about a color corresponding to a user, a color corresponding to an age group of the user, a color corresponding to each of emotions of the user, and a color corresponding to the acquaintance of the user as input through the application.

According to an embodiment, the processor 140 may be configured to extract RGB values of each of the colors, convert the RGB values into the three-dimensional color coordinates, and display the color coordinates on the application. The processor 140 may be configured to convert the extracted RGB values into L*, a*, and b* values, respectively, based on the Cielab conversion formula.

According to an embodiment, the processor 140 may be configured to calculate a distance between the color corresponding to the user and the color corresponding to each of emotions of the user on the three-dimensional color coordinates, apply the calculated distance to the three-dimensional color coordinates, and visually display the resulting three-dimensional color coordinates on the application.

According to an embodiment, when a color corresponding to each of emotions of the user is input, the processor 140 may be configured to calculate a distance between the color corresponding to the user and the color corresponding to each of emotions of the user, and may analyze a similarity between the color corresponding to the user and the color corresponding to each of emotions of the user, based on the calculated distance.

According to an embodiment, when the color corresponding to the user and the color corresponding to the user's age group are input from the user, the processor 140 may be configured to calculate a distance between the color corresponding to the user and the color corresponding to the user's age group, and analyze a similarity between the color corresponding to the user and the color corresponding to the user's age group based on the calculated distance.

According to an embodiment, when the color corresponding to each of emotions of the user and the color corresponding to the acquaintance of the user are input from the user, the processor 140 may be configured to calculate a distance between the color corresponding to each of emotions of the user and the color corresponding to the acquaintance of the user, and analyze a similarity between the color corresponding to each of emotions of the user and the color corresponding to the acquaintance, based on the calculated distance.

The similarity may be higher as the calculated distance is smaller, while the similarity may be lower as the calculated distance is larger. In addition, the similarity may be changed based on the number of the emotions input from the user. For example, as the number of emotions input from the user increases, the similarity based on the distance decreases, while as the number of emotions input from the user decreases, the similarity based on the distance may increase.

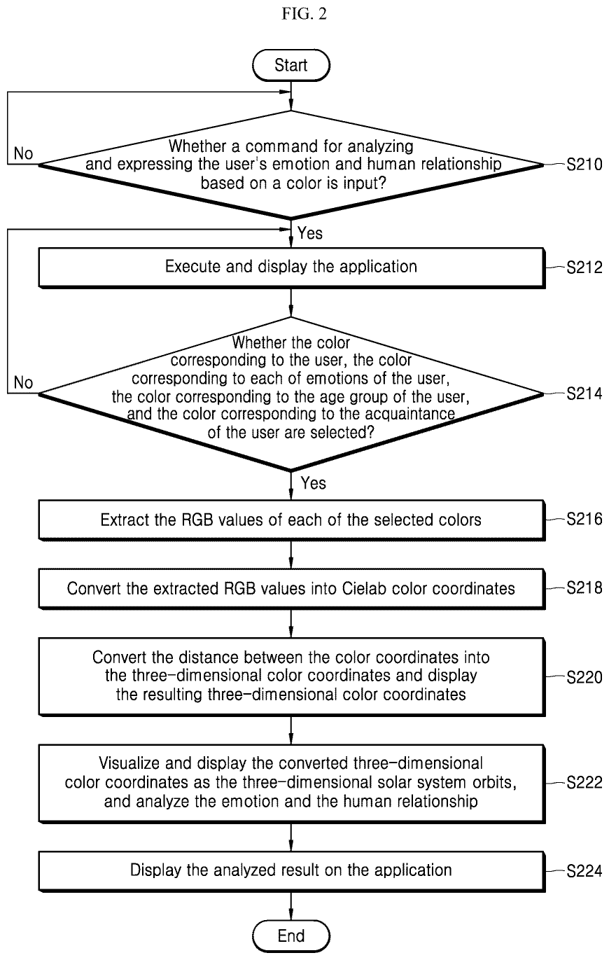

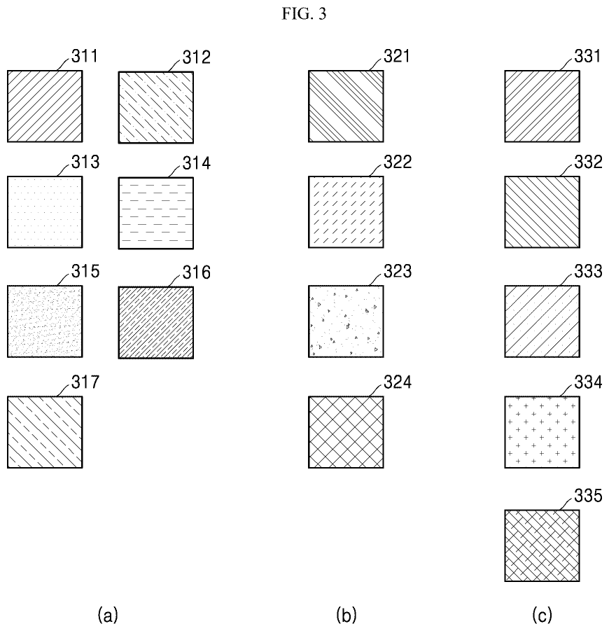

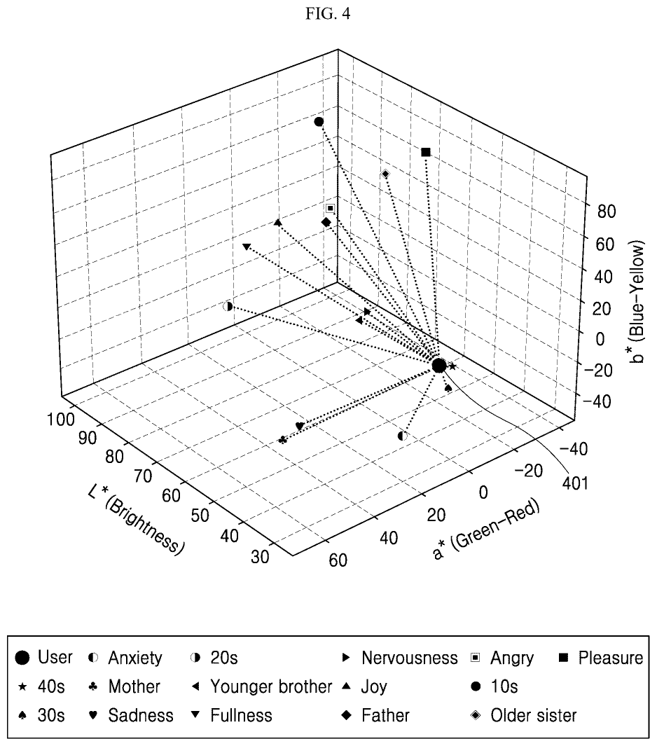

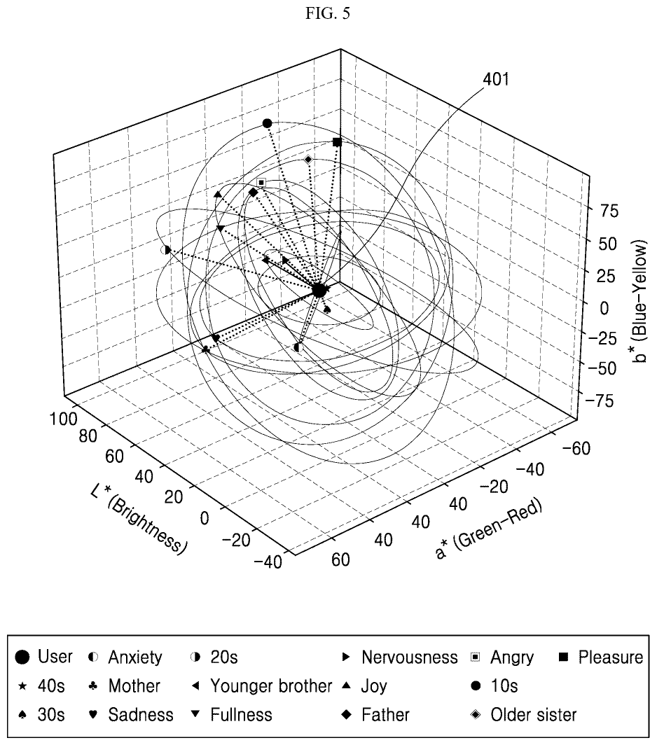

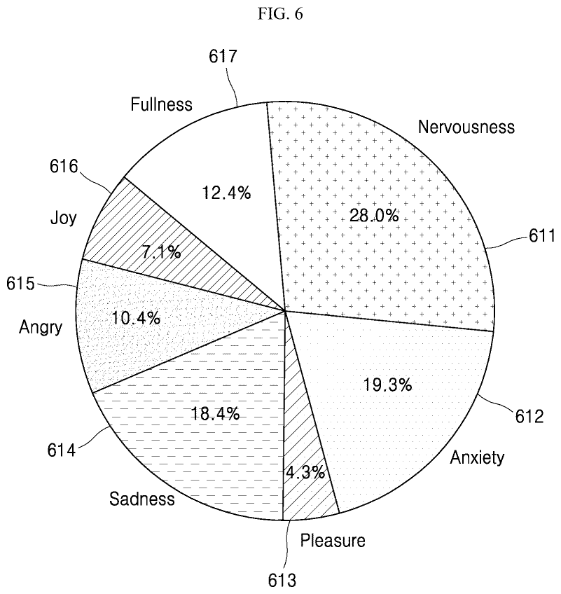

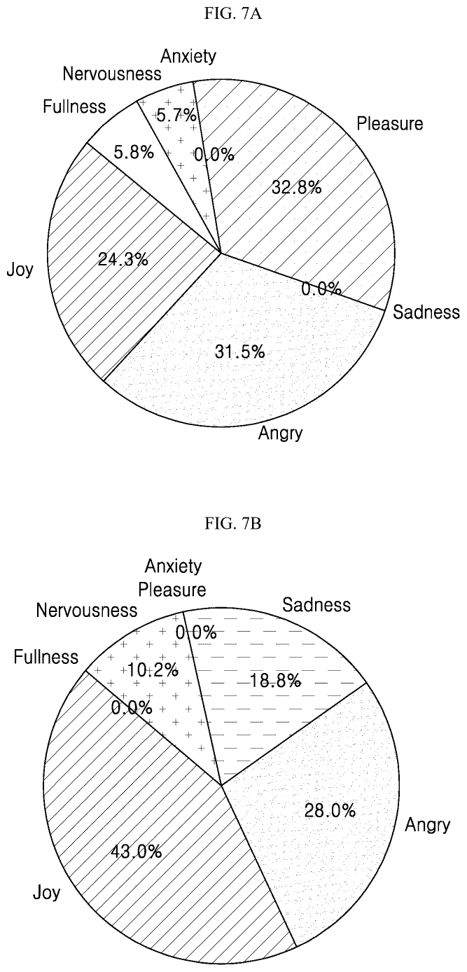

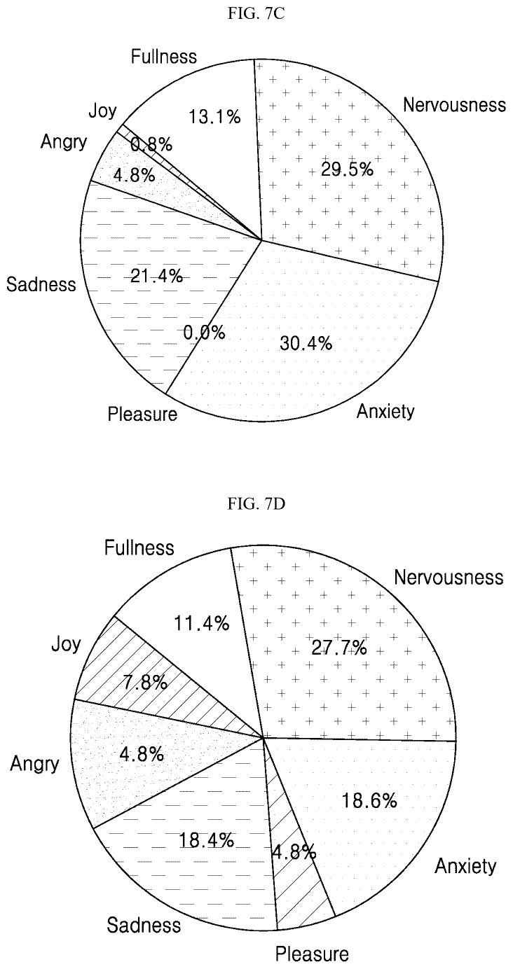

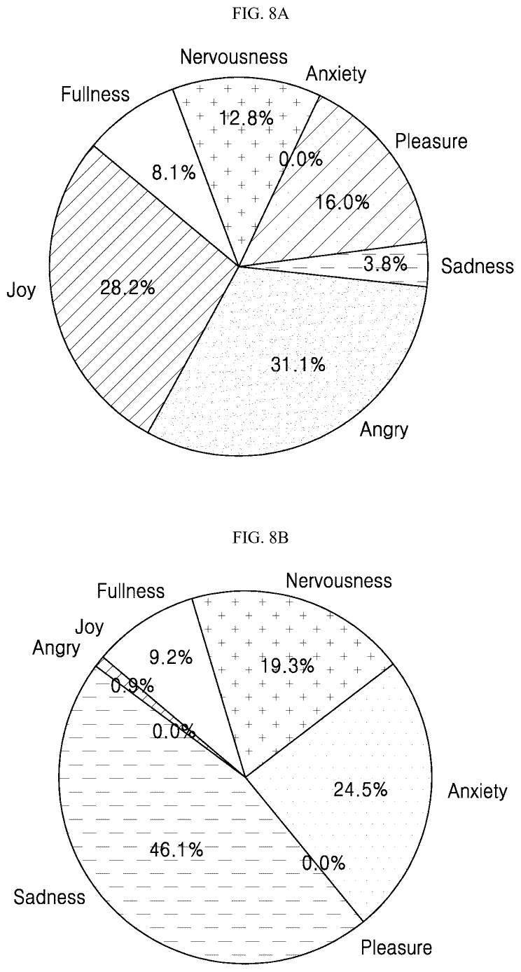

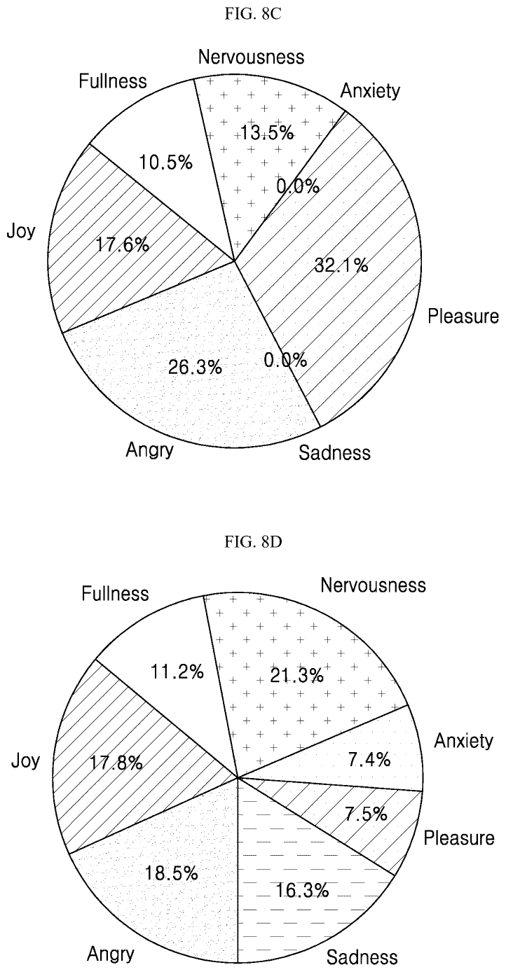

FIG. 2 is a flowchart illustrating a process of analyzing emotions and human relationships based on colors according to an embodiment of the present disclosure. (a) in FIG. 3 is an example diagram illustrating each of emotions of a user based on a color according to an embodiment of the present disclosure. (b) in FIG. 3 is an example diagram illustrating an age group of a user based on a color according to an embodiment of the present disclosure. (c) in FIG. 3 is an example diagram illustrating a color corresponding to an acquaintance of a user according to an embodiment of the present disclosure. FIG. 4 is an example diagram illustrating a three-dimensional color coordinate according to an embodiment. FIG. 5 is an example diagram illustrating a three-dimensional solar system orbits according to an embodiment of the present disclosure. FIG. 6 is an example diagram illustrating an emotion state of a user according to an embodiment of the present disclosure. FIG. 7A is an example diagram illustrating an emotion state of a user when the user is in his/her teens according to an embodiment of the present disclosure. FIG. 7B is an example diagram illustrating an emotion state of a user when the user is in his/her twenties according to an embodiment of the present disclosure. FIG. 7C is an example diagram illustrating an emotion state of a user when the user is in his/her thirties according to an embodiment of the present disclosure. FIG. 7D is an example diagram illustrating an emotion state of a user when the user is in his/her forties according to an embodiment of the present disclosure. FIG. 8A is an example diagram illustrating an emotion state of a user with respect to a father according to an embodiment of the present disclosure. FIG. 8B is an example diagram illustrating an emotion state of a user with respect to a mother according to an embodiment of the present disclosure. FIG. 8C is an example diagram illustrating an emotion state of a user with respect to an older sister according to an embodiment of the present disclosure. FIG. 8D is an example diagram illustrating an emotion state of a user with respect to a younger brother according to an embodiment of the present disclosure.

Hereinafter, a process of analyzing an emotion and a human relationship based on a color according to an embodiment of the present disclosure will be described in detail with reference to FIG. 2 to FIG. 8D.

According to an embodiment, the processor 140 may be configured to identify whether a command for analyzing and expressing the user's emotion and human relationship based on a color is input in S210. The processor 140 may be configured to identify whether a command for executing the application for analyzing and expressing the user's emotion and human relationship based on a color is input.

According to an embodiment, the processor 140 may be configured to execute and display the application in S212. When the command for executing the application for analyzing and expressing the user's emotion and human relationship based on the color is input, the processor 140 may be configured to execute the application and display the application on the display 110.

According to an embodiment, the processor 140 may be configured to identify whether the color corresponding to the user, the color corresponding to each of emotions of the user, the color corresponding to the age group of the user, and the color corresponding to the acquaintance of the user are selected in S214. The processor 140 may be configured to identify the color corresponding to the user, the color corresponding to each of emotions of the user, the color corresponding to the age group, and the color corresponding to the acquaintance of the user as selected or input by the user through the application.

For example, the user may input or select the color (i.e., a color corresponding to the user's temperament, personality, or disposition) of the user, the color corresponding to an age group (e.g., 10s, 20s, 30s, 40s, etc.) of the user, the color corresponding to each of emotions (e.g., joy, anger, sadness, pleasure, anxiety, novelty, fullness, etc.) of the user, and the color of the acquaintance of the user (e.g., father, mother, sister, brother, sister, sister, sister, brother, etc.) (i.e., a color corresponding to the temperament, personality, or disposition of the acquaintance) to or on the application through the input unit 120.

Referring to (a) of FIG. 3, the user may select a color determined as corresponding to each of his/her emotions (e.g., joy, anger, sadness, pleasure, anxiety, nervousness, fullness, etc.).

For example, the user may select a first color 311 determined as corresponding to joy, select a second color 312 determined as corresponding to anxiety, select a third color 313 determined as corresponding to anger, select a fourth color 314 determined as corresponding to nervousness, select a fifth color 315 determined as corresponding to sadness, select a sixth color 316 determined as corresponding to fullness, and select a seventh color 317 determined as corresponding to pleasure. (a) in FIG. 3 illustrates only seven colors corresponding to the emotions of the user. However, embodiments of the present disclosure are not limited thereto, the colors corresponding to the emotions of the users are not limited to the seven colors.

Referring to (b) in FIG. 3, the user may select the color corresponding to his/her age group.

For example, the user may select an eighth color 321 when he/she is in his/her 10s, select a ninth color 322 when he/she is in his/her 20s, select a tenth color 323 when he/she is in his/her 30s, and select an eleventh color 324 when he/she is in his/her 40s.

(b) in FIG. 3 shows only an example of the colors corresponding to the four age groups of the user. However, embodiments of the present disclosure are not limited thereto, and the colors corresponding to the age groups of the user are not limited to four colors, and may include the colors corresponding to the larger or smaller than 4 number of age groups of the user.

According to an embodiment, the processor 140 may be configured to calculate a distance between the color corresponding to each of emotions of the user selected in (a) in FIG. 3 and the color corresponding to the age group of the user selected in (b) in FIG. 3, and may apply the calculated distance to the three-dimensional color coordinates and visually display the resulting three-dimensional color coordinates on the application, so that the user may intuitively recognize a current state of various emotions based on the age group.

Referring to c of FIG. 3, the user may select the color corresponding to the user and the color corresponding to the acquaintance thereof.

For example, the user may select a color 331 corresponding to the user. When the user wants to express the relationship between himself or herself and his/her father based on a color, the user may select a color 332 corresponding to the father. In addition, when the user wants to express the relationship between himself or herself and his/her mother based on a color, the user may select a color 333 corresponding to the mother. In addition, when the user wants to express the relationship between himself and his/her older sister based on a color, the user may select a color 334 corresponding to the older sister. In addition, when the user wants to express the relationship between himself or herself and his/her younger brother based on a color, the user may select a color 335 corresponding to the younger brother.

(c) in FIG. 3 illustrates only the five colors corresponding to the five acquaintances. However, embodiments of the present disclosure are not limited thereto, and the colors corresponding to the acquaintances of the user are not limited to the five colors, and may include the colors corresponding to the larger or smaller than 5 number of acquaintances of the user.

According to an embodiment, the processor 140 may be configured to calculate a distance between the color corresponding to each of emotions of the user selected in (a) in FIG. 3 and the color corresponding to the acquaintance of the user selected in (c) in FIG. 3, may apply the calculated distance to the three-dimensional color coordinates, and may visually display the resulting three-dimensional color coordinates on the application, so that the user may intuitively recognize the current state of the emotions of the user with respect to the acquaintance.

According to an embodiment, the processor 140 may be configured to extract the RGB values of each of the selected colors in S216. The processor 140 may be configured to extract the RGB values of each of the color corresponding to the user (i.e., the color corresponding to the temperament, personality, or disposition of the user), the color corresponding to the user's age group, the color corresponding to each of the user's emotions, and the color corresponding to the user's acquaintance (i.e., the color corresponding to the temperament, personality, or disposition of the acquaintance) and store the extracted values in the memory 130. A following [Table 1] shows an example of the RGB values of each of the color corresponding to the user's emotion, the color corresponding to the user, the color corresponding to the age group of the user, and the color corresponding to the acquaintance of the user according to FIG. 3.

TABLE 1

| RGB values based on | RGB values based on age | RGB values based on user and |
|---|---|---|
| emotions | groups | acquaintances |
| Joy (RGB 230, 137, 15) | 10s (RGB 232, 222, 0) | User (RGB 80, 77, 78) |
| Anger (RGB 198, 154, 31) | 20s (RGB 228, 52, 17) | Father (RGB 195, 138, 15) |
| Sadness (RGB 160, 54, 118) | 30s (RGB 52, 76, 102) | Mother (RGB 166, 56, 139) |
| Pleasure (RGB 132, 186, 5) | 40s (RGB 73, 68, 64) | Sister (RGB 164, 186, 51) |
| Anxiety (RGB 10, 93, 171) | | Brother (RGB 148, 89, 57) |
| Nervousness (RGB 142, 140, | | |
| 134) | | |
| Fullness (RGB 255, 255, | | |
| 255) | | |

The RGB values refer to pixel-based color data having a range of 0 to 255.

According to an embodiment, the processor 140 may be configured to convert the extracted RGB values into Cielab color coordinates in S218. The processor 140 may be configured to convert the extracted RGB values into L*, a*, and b* values according to a Cielab conversion formula (e.g., a RGB2lab function). In this case, the processor 140 may be configured to convert each of all colors into the Cielab values while using the Cielab value corresponding to the color corresponding to the user as a reference point (0, 0, and 0).

According to an embodiment, the processor 140 may be configured to convert the distance between the color coordinates into the three-dimensional color coordinates and display the resulting three-dimensional color coordinates in S220. The processor 140 may be configured to calculate the distance between the color coordinates using a following [Equation 1]. In addition, the processor 140 may be configured to calculate a distance between the reference point (0, 0, 0) and each of other color coordinates on the three-dimensional color coordinates.

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} \qquad [ Equation 1 ]$$

In the above [Equation 1], L* represents a brightness difference, a* represents a difference between green and red, and b* represents a difference between blue and yellow.

In addition, the processor 140 may be configured to respectively correspond the X, Y, and Z axes of the three-dimensional space to the L*, a*, and b* values, and then visualize the converted Cielab color coordinates in the three-dimensional space based on a ΔE value (i.e., ΔE distance) and display the visualized color coordinates on the application.

The ΔE value (ΔE distance) refers to a value obtained by calculating the distance between the reference point and each of other color coordinates in the form of the Cielab value using the ΔE Distance formula. A similarity between the reference point (0, 0, 0) and other color coordinates may be quantified as the distance (ΔE Distance) and may be used for the visualization setting.

The ΔE value refers to the distance between coordinates. Thus, the smaller the value, the smaller the color difference, while the larger the value, the larger the color difference.

For example, a low ΔE value means that the brightness (L*) values of the two colors are similar to each other and the hue (a*, b*) values of the two colors are similar to each other. This means that the two colors look similar to each other even when being viewed with the human eye.

A following Table 2 shows an example of converting the RGB values of each of the color corresponding to the user's emotion, the color corresponding to the user, the color corresponding to the age group of the user, and the color corresponding to the acquaintance of the user according to FIG. 3 into the Cielab values.

TABLE 2

| Cielab values based on | Cielab values based on age | Cielab values based on user |
|---|---|---|
| emotions | groups | and acquaintances |
| Joy (Cielab 32.50, 26.85, | 10s (Cielab 53.65, −16.64, | User (Cielab 0.00, 0.00, |
| 69.31) | 86.19) | 0.00) |
| Anger (Cielab 32.86, 4.16, | 20s (Cielab 17.63, 63.71, | Father (Cielab 28.44, 11.21, |
| 64.26) | 58.72) | 64.42) |
| Sadness (Cielab 7.23, | 30s (Cielab −1.58, −2.71, −17.85) | Mother (Cielab 9.41, |
| 48.76, −13.25) | 40s (Cielab −3.84, −0.16, 3.33) | 52.63, −22.72) |
| Pleasure (Cielab | | Sister (Cielab 38.67, −26.49, |
| 36.44, −40.85, 69.62) | | 62.03) |
| Anxiety (Cielab 6.24, | | Brother (Cielab 10.80, |
| 6.70, −47.76) | | 19.63, 28.74) |
| Nervousness (Cielab | | |
| 25.20, −1.85, 3.61) | | |
| Fullness (Cielab | | |
| 66.94, −1.45, 0.15) | | |

The Cielab values are composed of L* (brightness), a* (green-red axis), b* (blue-yellow axis), and refer to color data reconstructed by applying the human visual sensation to the RGB color.

Referring to FIG. 4, the processor 140 may be configured to display the distance calculated based on the above [Table 2] as three-dimensional color coordinates on the application. The processor 140 may be configured to calculate the distance between each of the color corresponding to the user's emotion, the color corresponding to the age group of the user, and the color corresponding to the acquaintance of the user and a reference point 401 as the Cielab values (0.00, 0.00, 0.00) of the color corresponding to the user using the above [Equation 1], and may display each of the calculated distances as the three-dimensional color coordinates as shown in FIG. 4.

According to an embodiment, the processor 140 may be configured to visualize and display the converted three-dimensional color coordinates as the three-dimensional solar system orbits, and analyze the emotion and the human relationship in S222. The processor 140 may be configured to visualize the converted three-dimensional color coordinates as the three-dimensional solar system orbits (i.e., a state in which each of the planets revolves around the sun) and display the visualized three-dimensional solar system orbits on the application.

Referring to FIG. 5, the processor 140 may be configured to convert the three-dimensional color coordinates of each of the color corresponding to the user's emotion, the color corresponding to the age group of the user, and the color corresponding to the acquaintance of the user into the three-dimensional solar system orbits (i.e., a state in which each of the planets orbits around the sun) using the Cielab values according to the above [Table 2] and using the reference point 401 as the Cielab values (0.00, 0.00, 0.00) of the color corresponding to the user. Then, the processor 140 may be configured to display the converted three-dimensional solar system orbits on the application. For example, the center of the orbits refers to the reference point corresponding to the color corresponding to the user.

According to an embodiment, the processor 140 may be configured to display the analyzed result on the application in S224. The processor 140 may be configured to display, on the application, a result of analyzing the emotion of the user, the age group of the user, and the relationship with the acquaintance, based on the colors and the distances (i.e., the distance from the center point).

Referring to FIG. 6, the processor 140 may be configured to analyze the similarity between the color corresponding to the user and the color corresponding to each of the emotions such as anxiety, nervousness, pleasure, sadness, anger, joy, fullness, etc., and visually display the similarity on the application.

For example, a similarity 611 between the color corresponding to the user and the color corresponding to the nervousness is 28.0%. Thus, the user may identify that the tense situation of the user continues, and the stress level of the user is high, and the emotion is highly unstable.

For example, a similarity 612 between the color corresponding to the user and the color corresponding to the anxiety is 1⁹0.3%. Thus, the user may identify that the anxiety and nervousness are likely to be the main emotions at present.

For example, a similarity 613 between the color corresponding to the user and the color corresponding to the pleasure is 4.3%. Thus, the user may identify that the user has little pleasure or satisfaction or has low satisfaction in daily life.

For example, a similarity 614 between the color corresponding to the user and the color corresponding to the sorrow is 18.4%. Thus, the user may identify that the current emotional state is related to the sadness, and that the emotion is heavy and dark.

For example, a similarity 615 between the color corresponding to the user and the color corresponding to the anger is 10.4%. Thus, the user may identify that the user is not in a state of being greatly negative or angry, and has a conflict or complaint which is not at a serious level.

For example, a similarity 616 between the color corresponding to the user and the color corresponding to the joy is 7.1%. Thus, the user may identify that the feeling of joy is insufficient, and thus there is very little positive energy in life.

For example, a similarity 617 between the color corresponding to the user and the color corresponding to the fullness is 12.4%. Thus, the user may identify that the heart is not full, but the deficiency feeing is more prominent in the heart.

As described above, when the user selects the color corresponding to the user and the color corresponding to each of the emotions such as anxiety, nervousness, pleasure, sadness, anger, joy, and fullness, the processor 140 may be configured to analyze the similarity between the selected color corresponding to the user and the selected color corresponding to each of the emotions such as anxiety, nervousness, pleasure, sadness, anger, joy, and fullness, and visually display the similarity on the application, so that the user may intuitively grasp the current state of the user related to the various emotions.

The processor 140 may be configured to analyze the similarity between the color corresponding to the age group selected by the user and the selected color corresponding to each of the emotions such as anxiety, nervousness, pleasure, sadness, anger, joy, and fullness selected by the user and visually display the similarity on the application.

Referring to FIG. 7A, the user in teens is energetic, and a similarity between the color corresponding to each of sadness and anxiety and the color corresponding to the teens is zero, and thus the user is emotionally stable, and is far from the sadness.

Referring to FIG. 7B, it may be identified that a similarity between the color corresponding to the age group 20s and the color corresponding to each of joy and anger is high. In particular, it may be identified that a similarity between the color corresponding to the age group 20s and the color corresponding to the nervousness is 10.2% and thus the user is a little nervous.

Referring to FIG. 7C, it may be understood that the user is in his or her 30s and a similarity between the color corresponding to the age group 30s and the color corresponding to the joy is very low, and a similarity between the color corresponding to the age group 30s and the color corresponding to each of anxiety and nervousness is high. Thus, the stress and worry are the main emotions of the user. A similarity between the color corresponding to the age group 30s and the color corresponding to the sadness is high. Thus, the user has a heavy responsibility feeling.

Referring to FIG. 7D, it may be identified that the user is in his or her 40s and a similarity between the color corresponding to the age group 40s and the color corresponding to each of anxiety and nervousness is high. Thus, the stress and worry are the main emotions of the user. A similarity between the color corresponding to the age group 40s and the color corresponding to each of the joy and angry has a middle level.

As described above, when the user selects the color corresponding to the age group and the color corresponding to each of the emotions such as anxiety, nervousness, pleasure, sadness, anger, joy, and fullness, the processor 140 may be configured to calculate the similarity between the color corresponding to the age group selected by the user and the selected color corresponding to each of the emotions such as anxiety, nervousness, pleasure, sadness, anger, joy, and fullness selected by the user and visually display the similarity on the application. Thus, the user may intuitively grasp the current state of the user with respect to various emotions based on his/her age group.

The processor 140 may be configured to analyze a similarity between the selected color corresponding to each of the emotions such as anxiety, nervousness, pleasure, sadness, anger, joy, and fullness selected by the user and the color corresponding to his or her acquaintance selected by the user and visually display the similarity on the application.

Referring to FIG. 8A, the user has a very high similarity level between the color corresponding to each of the joy and anger and the color corresponding to the father. Thus, it may be identified that the user has a complex feeling including both pleasure and conflict relative to the father of the user. Thus, it may be determined that the relationship of the user with the father may be positive and at the same time, a conflict relative to the father is strong. In addition, the user has a very low similarity level between the color corresponding to each of the sadness and the color corresponding to the father. Further, the user has a middle similarity level between the color corresponding to the nervousness and the color corresponding to the father.

Referring to FIG. 8B, the user has a very high similarity level between the color corresponding to the sadness and the color corresponding to the mother. Thus, it may be identified that the relationship of the user relative to the mother is emotionally heavy and burdensome. In addition, the user has a very low similarity level between the color corresponding to each of the joy and anger and the color corresponding to the mother. Thus, there is little positive feeling exchange between the mother and the user. Further, the user has a middle similarity level between the color corresponding to each of the nervousness and the anxiety and the color corresponding to the mother. Thus, the user may grasp that the relationship with the mother has difficulty and may act as a stressor.

Referring to FIG. 8C, the user has a very high similarity level between the color corresponding to each of the pleasure and anger and the color corresponding to the older sister. Thus, it may be identified that the relationship of the user relative to the older sister is a complex relationship in which pleasure and conflict are repeated. In addition, the user has a middle similarity level between the color corresponding to the joy and the color corresponding to the older sister. Further, the user has the very low similarity level between the color corresponding to the anxiety and the color corresponding to the older sister. Further, the user has the middle similarity level between the color corresponding to the nervousness and the color corresponding to the older sister. Thus, it may be understood that a tense situation with respect to the older sister sometimes occurs.

Referring to FIG. 8D, the user may recognize that various emotions are intricately intertwined in the relationship between the user and the younger brother, and the user has balanced joy and anger, and has significantly increased sadness in the relationship between the user and the younger brother, thus indicating that the emotional fluctuation with respect to the younger brother is large. In addition, the user often has a quarrel with the younger brother, but at the same time, the user has a lot of time to enjoy with the younger brother. The user has a very high similarity level between the color corresponding to the nervousness and the color corresponding to the younger brother. Thus, it is understood that tension and competition are often felt in the relationship with the younger brother.

As described above, when the user selects the color corresponding to the acquaintance and the color corresponding to each of the emotions such as anxiety, nervousness, pleasure, sadness, anger, joy, and fullness, the processor 140 may be configured to calculate the similarity between the color corresponding to the acquaintance selected by the user and the color corresponding to each of the emotions such as anxiety, nervousness, pleasure, sadness, anger, joy, and fullness selected by the user and visually display the similarity on the application. Thus, the user may intuitively grasp the current state of various emotions of the user with respect to the acquaintance.

In addition, the method and device of the present disclosure may visually present the emotion, the psychological state based on the age group, and the human relationship using the three-dimensional colors coordinates.

In addition, the method and device of the present disclosure may visually present the emotion, the psychological state based on the age group, and the human relationship in the solar system orbital form in which the color corresponding to the user acts as the center point, and each color as each planet orbits the sun as the center point, such that the user may intuitively recognize the emotion, the psychological state based on the age group, and the human relationship, thereby providing visual information so that the user may understand in various ways how the emotion, the psychological state based on the age group, and the human relationship relate to each other.

The steps or operations in each of the flowcharts as described above may be executed regardless of the illustrated order, or may be executed simultaneously. In addition, at least one component of the device of the present disclosure and at least one operation performed by the at least one component may be implemented in hardware and/or software.

Although the embodiments of the present disclosure have been described in more detail with reference to the accompanying drawings, the present disclosure is not necessarily limited to the embodiments, and may be modified in a various manner within the scope of the technical spirit of the present disclosure. Accordingly, the embodiments as disclosed in the present disclosure are intended to describe rather than limit the technical idea of the present disclosure, and the scope of the technical idea of an embodiment of the present disclosure is not limited by these embodiments. Therefore, it should be understood that the embodiments described above are not restrictive but illustrative in all respects. In addition, even though the effects according to the configuration of the present disclosure are not explicitly described while illustrating the embodiment of the present disclosure, it is obvious that the predictable effect therefrom should also be recognized.

What is claimed is:

1. A device for analyzing an emotion and a human relationship based on a color, the device comprising:
a display; and
a processor electrically connected to the display,
wherein the processor is configured to:
display an application for analyzing an emotion and a human relationship based on a color on the display;
identify a color corresponding to a user, a color corresponding to an age group of the user, a color corresponding to each of emotions of the user, and a color corresponding to an acquaintance of the user as input from the user through the displayed application;
extract RGB values of each of the identified colors, convert the extracted RGB values into three-dimensional color coordinates, and display the converted three-dimensional color coordinates on the application;
convert the converted three-dimensional color coordinates into three-dimensional solar system orbits and display the converted three-dimensional solar system orbits on the application; and
analyze the user's emotion, the age group of the user, and a relationship between the user and the acquaintance, based on the converted three-dimensional solar system orbits and display the analyzed result on the display.

2. The device of claim 1, wherein the processor is further configured to convert the extracted RGB values into L*, a*, and b* values, respectively, based on a Cielab conversion formula.

3. The device of claim 2, wherein the processor is further configured to:
calculate a distance between the color corresponding to the user and the color corresponding to each of emotions of the user, on the three-dimensional color coordinates; and
apply the calculated distance to the three-dimensional color coordinates and visually display the resulting three-dimensional color coordinates on the application.

4. The device of claim 3, wherein the processor is further configured to:
when the color corresponding to the user and the color corresponding to each of emotions are input from the user, calculate a distance between the color corresponding to the user and the color corresponding to each of emotions of the user; and
analyze a similarity between the color corresponding to the user and the color corresponding to each of emotions of the user, based on the calculated distance.

5. The device of claim 3, wherein the processor is further configured to:
when the color corresponding to each of emotions of the user and the color corresponding to the age group of the user are input from the user, calculate a distance between the color corresponding to each of emotions of the user and the color corresponding to the age group of the user; and
analyze a similarity between the color of each of emotions of the user and the color corresponding to the age group of the user, based on the calculated distance.

6. The device of claim 3, wherein the processor is further configured to:
when the color corresponding to each of emotions of the user and the color corresponding to the acquaintance are input from the user, calculate a distance between the color corresponding to each of emotions of the user and the color corresponding to the acquaintance; and
analyze a similarity between the color corresponding to each of emotions of the user and the color corresponding to the acquaintance, based on the calculated distance.

7. The device of claim 4, wherein as the calculated distance is smaller, the similarity is higher,
wherein as the calculated distance is greater, the similarity is lower.

8. The device of claim 5, wherein as the calculated distance is smaller, the similarity is higher,
wherein as the calculated distance is greater, the similarity is lower.

9. The device of claim 6, wherein as the calculated distance is smaller, the similarity is higher,
wherein as the calculated distance is greater, the similarity is lower.

10. A method for analyzing an emotion and a human relationship based on a color, the method comprising:
displaying an application for analyzing an emotion and a human relationship based on a color on a display;
identifying a color corresponding to a user, a color corresponding to an age group of the user, a color corresponding to each of emotions of the user, and a color corresponding to an acquaintance of the user as input from the user through the displayed application;
extracting RGB values of each of the identified colors, converting the extracted RGB values into three-dimensional color coordinates, and displaying the converted three-dimensional color coordinates on the application;
converting the converted three-dimensional color coordinates into three-dimensional solar system orbits and displaying the converted three-dimensional solar system orbits on the application; and
analyzing the user's emotion, the age group of the user, and a relationship between the user and the acquaintance, based on the converted three-dimensional solar system orbits and displaying the analyzed result on the display.

11. The method of claim 10, wherein extracting the RGB values each of the identified colors, converting the extracted RGB values into the three-dimensional color coordinates, and displaying the converted three-dimensional color coordinates on the application include:
calculating a distance between the color corresponding to the user and the color corresponding to each of emotions of the user, on the three-dimensional color coordinates; and
applying the calculated distance to the three-dimensional color coordinates and visually displaying the resulting three-dimensional color coordinates on the application.

12. The method of claim 11, wherein analyzing the user's emotion, the age group of the user, and the relationship between the user and the acquaintance includes:
when the color corresponding to the user and the color corresponding to each of emotions are input from the user, calculating a distance between the color corresponding to the user and the color corresponding to each of emotions of the user; and
analyzing a similarity between the color corresponding to the user and the color corresponding to each of emotions of the user, based on the calculated distance.

13. The method of claim 11, wherein analyzing the user's emotion, the age group of the user, and the relationship between the user and the acquaintance includes:
when the color corresponding to each of emotions of the user and the color corresponding to the age group of the user are input from the user, calculating a distance between the color corresponding to each of emotions of the user and the color corresponding to the age group of the user; and
analyzing a similarity between the color of each of emotions of the user and the color corresponding to the age group of the user, based on the calculated distance.

14. The method of claim 11, wherein analyzing the user's emotion, the age group of the user, and the relationship between the user and the acquaintance includes:
when the color corresponding to each of emotions of the user and the color corresponding to the acquaintance are input from the user, calculating a distance between the color corresponding to each of emotions of the user and the color corresponding to the acquaintance; and
analyzing a similarity between the color corresponding to each of emotions of the user and the color corresponding to the acquaintance, based on the calculated distance.

15. A computer program stored in a computer readable medium, wherein the computer program stores therein instructions for analysis of an emotion and a human relationship based on a color,
wherein when the instructions are executed by a processor, the instructions cause the processor to:
display an application for analyzing an emotion and a human relationship based on a color on a display;
identify a color corresponding to a user, a color corresponding to an age group of the user, a color corresponding to each of emotions of the user, and a color corresponding to an acquaintance of the user as input from the user through the displayed application;
extract RGB values of each of the identified colors, convert the extracted RGB values into three-dimensional color coordinates, and display the converted three-dimensional color coordinates on the application;
convert the converted three-dimensional color coordinates into three-dimensional solar system orbits and display the converted three-dimensional solar system orbits on the application; and
analyze the user's emotion, the age group of the user, and a relationship between the user and the acquaintance, based on the converted three-dimensional solar system orbits and display the analyzed result on the display.
